(19) United States
(12) Patent Application Publication
(10) Pub. No.: US 2018/0028078 A1
(43) Pub. Date: Feb. 1, 2018

(54) CONTINUOUS AND RAPID QUANTIFICATION OF STROKE VOLUME FROM MAGNETOHYDRODYNAMIC VOLTAGES IN MAGNETIC RESONANCE IMAGING

(71) Applicants: The Brigham and Women's Hospital, Inc., Boston, MA (US); University of Georgia Research Foundation, Inc., Athens, GA (US)

(72) Inventors: T. Stanley Gregroy, Burlington, NC (US); Tsz Ho Tse, Lawrenceville, GA (US); Ehud J. Schmidt, Newton, MA (US)

(21) Appl. No.: 15/548,828

(22) PCT Filed: Feb. 5, 2016
(86) PCT No.: PCT/US16/16770

§ 371 Date: Aug. 4, 2017

Related U.S. Application Data
(60) Provisional application No. 62/112,733, filed on Feb. 6, 2015.

(51) Int. Cl. A61B 5/026; A61B 5/00; A61B 5/0408; A61B 5/04; A61B 5/055; A61B 5/029

(52) U.S. Cl. CPC A61B 5/0263; A61B 5/055; A61B 5/029; A61B 5/0408; A61B 5/04011; A61B 5/7278; A61B 5/7253

(57) ABSTRACT

Described here are systems and methods for providing a non-invasive and continuous quantitative measurement of left ventricular stroke volume (“SV”) and flow volume during a magnetic resonance imaging (“MRI”) scan. In general, the method estimates quantitative measurements of SV from magnetohydrodynamic (“MHD”] voltages generated by blood flowing through the subject's vasculature while the subject is positioned in the magnetic field of an MRI system. A rapid calibration technique is provided to convert MHD voltages to estimates of blood flow, from which quantitative measurements of SV can be computed.

### CROSS-REFERENCE TO RELATED APPLICATIONS

This application claims the benefit of U.S. Provisional Patent Application Ser. No. 62/112,733, filed on Feb. 6, 2015, and entitled “Systems and Methods for Measuring Ventricular Flow and Stroke Volume.”

### STATEMENT REGARDING FEDERALLY SPONSORED RESEARCH

This invention was made with government support under EB013873 awarded by the National Institutes of Health. The government has certain rights in the invention.

### BACKGROUND OF THE INVENTION

The field of the invention is systems and methods for physiological monitoring during a magnetic resonance imaging (“MRI”) scan, including the continuous and real-time monitoring of blood flow and left ventricular stroke volume from magnetohydrodynamic voltage measurements.

Left ventricular stroke volume (SV) is a measure of the amount of blood ejected into the aortic arch during the systolic phase of the cardiac cycle. Real-time or beat-to-beat SV, measured over successive cardiac cycles, is commonly used to evaluate left-ventricular (LV) mechanical function, which can detect pathological response to stress and arrhythmias.

When the human body needs to exert a larger effort (i.e., when it is placed in stress), the body has to pump blood at a greater rate to meet the larger demand for oxygen and glucose by tissues. In these instances, the body responds by increasing the stroke volume (e.g., by increasing heart rate), which can persist until a certain degree of effort, which is called the point of peak stress.

In patients with cardiac diseases, such as those resulting from injury to the muscle of the LV (ischemic disease), or those suffering from cardiac rhythm problems (arrhythmia), increasing the stress level can lead to pathological conditions. One possible result of increased stress is an increase in irregular heart beats, such as Premature Ventricular Contractions (PVCs), which are a type of inefficient beats that generate very little stroke volume. Another possible outcome is the appearance of all irregular form of electrical signal in the cardiac muscle, which can lead to a cardiac event (such as a heart attack). In all cases, it is important to detect the onset of such cardiac events quickly, since if the response is not sufficiently fast, such as through the delivery of medication, cardiopulmonary resuscitation, or defibrillation, the brain, the heart, or other tissues can be damaged, possibly leading to death.

As a result, the American Heart Association and the American Association of Anesthesiologists have defined specific surgical and interventional procedures for which, in higher-risk patient populations, they recommend continuously monitoring patients for changes in stroke volume (or cardiac output). Current methods for monitoring these patients involve the use of Invasive Blood Pressure (IBP) probes, which are pressure-measuring catheters placed into the aorta, or Transesophageal Echo (TEE) probes, which are ultrasound probes lowered into the esophagus to a level that is adjacent to the heart. Both of these methods work well, but require invasive procedures that carry associated risks (e.g., perforation of the aortic wall, esophagus, or aorta).

There are a few non-invasive measures of SV, the most common one being measurement of SPO2 (oxygen saturation) in the fingers, but SPO2 has been shown to be insufficiently sensitive to detect cardiac events in a timely manner. The best non-invasive tool to measure SV is to perform Phase-Contrast (PC) MRI imaging of flow coming out of the LV (i.e., by measuring the flow just about the aortic valve). However, because the MRI scanner is used for imaging a variety of contrasts and/or anatomic regions during an imaging session, it's use to continuously measure SV is not a viable (or cost effective) solution.

Thus, there remains a need for systems and methods capable of providing a non-invasive quantitative measure of stroke volume and flow volume for use in patients who are placed in an MRI scanner for imaging or for performance of MR-guided interventions. In the absence of such tools, which could be used to reliably measure patient well-being inside an MRI scanner, large patient populations are excluded from MRI or MRI-guided procedures.

### SUMMARY OF THE INVENTION

The present invention overcomes the aforementioned drawbacks by providing a method for computing a measurement of left ventricular stroke volume from a subject positioned in a bore of a magnetic resonance imaging (MRI) system. The method includes recording electrocardiogram (ECG) measurements from a subject positioned in a bore of an MRI system. Magnetohydrodynamic voltage (VMHD) measurements are estimated from the ECG measurements, and a VMHD vector is generated by converting the VMHD measurements to a vectorcardiogram reference frame. Calibration data is generated by correlating the VMHD vector to a standard measure of blood flow obtained from the subject. A VMHD-based blood flow measurement is then generated by converting the VMHD vector to the VMHD-based blood flow measurement using the calibration data, and a stroke volume measurement is computed from the VMHD-based blood flow measurement.

It is another aspect of the invention to provide a method for providing continuous real-time monitoring of left ventricular stroke volume in a subject positioned in a bore of an MRI system. The method includes providing a calibrated multiple-parameter linear regression (MLR) model to a computer system. The MLR model includes subject-specific coefficients that relate magnetohydrodynamic voltage (VMHD) vectorcardiogram components to blood flow as a function of time. ECG measurements are recorded from a subject positioned in a bore of an MRI system and VMHD measurements are estimated from the ECG measurements. VMHD vectorcardiogram components are generated by converting the VMHD measurements to a vectorcardiogram reference frame, and VMHD-based blood flow measurements are generated by inputting the VMHD vectorcardiogram components to the calibrated MLR model. Stroke volume measurements can then be computed from the VMHD-based blood flow measurements, thereby providing continuous real-time monitoring of left ventricular stroke volume in the subject while the subject is positioned in the bore of the MRI system.

The foregoing and other aspects and advantages of the invention will appear from the following description. In the description, reference is made to the accompanying drawings that form a part hereof, and in which there is shown by way of illustration a preferred embodiment of the invention. Such embodiment does not necessarily represent the full scope of the invention, however, and reference is made therefore to the claims and herein for interpreting the scope of the invention.

### BRIEF DESCRIPTION OF THE DRAWINGS

FIG. 1A is an example set of electrocardiogram (“ECG”) traces acquired from a subject who is not positioned in a magnetic resonance imaging (“MRI”) system. In this figure, V1-V6 refer to signals obtained from different electrodes placed on the human chest using a conventional 12-lead ECG system.

FIG. 1B is an example set of ECG traces acquired from a subject who is positioned in an MRI system, and which shows effects of magnetohydrodynamic voltages (“VMHD”) generated in the ECG electrodes by the MRI system.

FIG. 2 is a flowchart setting forth the steps of an example method for computing blood flow and stroke volume measurements from VMHD measurements.

FIG. 3A is an example vectorcardiogram acquired from a subject who is not positioned in an MRI system.

FIG. 3B is an example vectorcardiogram acquired from a subject who is positioned in an MRI system, and which shows spatial components of the vectorcardiogram associated with VMHD.

FIG. 4 is a block diagram of an example of an MRI system.

FIG. 5 depicts example plots of VMHD vectorcardiogram spatial components (VMHD_{X}, VMHD_{Y}, VMHD_{Z}) and MRI-based and VMHD-based estimates of blood flow.

FIG. 6 depicts example plots of VMHD-based blood flow and stroke volume measurements in addition to heart rate and an ECG trace from the V6 electrode.

### DETAILED DESCRIPTION OF THE INVENTION

Described here are systems and methods for providing a non-invasive and continuous quantitative measurement of left ventricular stroke volume (“SV”) and flow volume during a magnetic resonance imaging (“MRI”) scan. In general, the method estimates quantitative measurements of SV from magnetohydrodynamic (“MHD”) voltages generated by blood flowing through the subject's vasculature while the subject is positioned in the magnetic field of an MRI system.

Blood flowing out from the left ventricle flows into the ascending aorta and, thereafter, into the aortic arch. From the aortic arch, the blood flow splits into several arteries that feed various regions and tissues in the body. As a result, measuring blood flow in the aortic arch provides a very good estimate of SV.

In MRI scanners, the subject is placed in a strong static magnetic field often referred to as the main magnetic field, B₀. Because blood plasma contains salt ions, when blood flows in the main magnetic field of an MRI system, an electrical voltage is created according to Lorentz's law. This voltage is referred to as the MHD voltage, VMHD. This voltage is can be estimated as,

$$VMHD ∝ {\int }_{A} v \times {B}_{0} ; \qquad ( 1 )$$

where v is the blood flow velocity, B₀ is the main magnetic field, A is the cross-sectional area of the blood vessel through which the blood is flowing, and “×” denotes the cross product operator. Thus, VMHD is largest when the flow velocity is rapid, the vessel cross-section is large, and the flow is oriented perpendicular to the magnetic field direction.

When the human heart is placed in the center of the MRI magnet, such as occurs when cardiac MRI is performed, VMHD is dominated entirely by flow in the aortic arch because flow in the arch is fast (e.g., around 1.5 m/s), the aortic diameter is large (e.g., about 1 cm), and the aortic arch is oriented perpendicular to the main magnetic field direction (i.e., B₀ is oriented along the longitudinal axis and thus lies along the length of the subject's body).

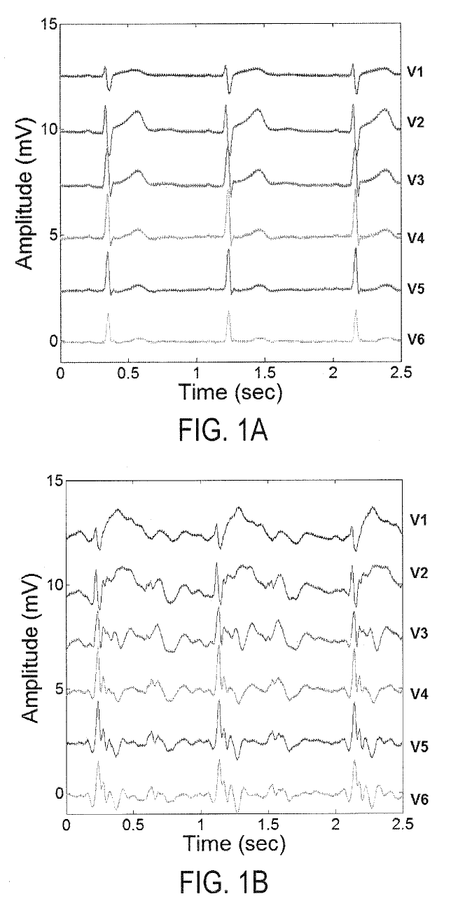

When electrocardiograms (“ECGs”) are measured inside an MRI scanner, the resulting ECG traces differ from their usual appearance outside the MRI system (the so-called “real” ECG trace) because the traces acquired while the subject is in the MRI scanner contain an additional component that is due to VMHD. In high-field MRI systems (e.g., those having B₀ of 3 T or greater), the VMHD component in an ECG trace can be stronger than the real ECG component. An example of ECG traces acquired outside of an MRI system are illustrated in FIG. 1A, and an example of ECG traces acquired from the same subject while positioned in an MRI system are illustrated in FIG. 1B.

Because VMHD tends to mask the true ECG, which complicates seeing several segments of the real ECG as required to detect changes in the ECG, multiple methods to remove the VMHD component have been developed. Moreover, because VMHD depends on flow in the aortic arch, it has been known to correlate with the flow out of the left ventricle, which also suggests that it is correlated with SV. However, a quantitative measure of SV cannot be computed or estimated directly from the VMHD measurements because of at least two reasons. One reason is because the relationship between VMHD and SV depends strongly on the specific orientation of the aortic arch in the main magnetic field, B₀, of the MRI system and this orientation influences the size of the VMHD seen in any given ECG electrode. Another reason is because the shape and dimensions of a particular subject's torso will influence the measurement of VMHD because the shape and dimensions of the torso influence the electrical current flow from the heart to the surface.

The systems and methods of the present invention overcome these limitations by providing a quantitative relationship between VMHD and SV. This quantitative relationship is generally based on converting the individual VMHD traces into a vectorial VMHD form, which has only three spatial components. This vectorial VMHD measurement can then be rapidly calibrated against a standard measurement of blood flow, such as a measurement of blood flow from phase contrast cine MRI. Real-time estimates of SV can then be produced based on this calibration.

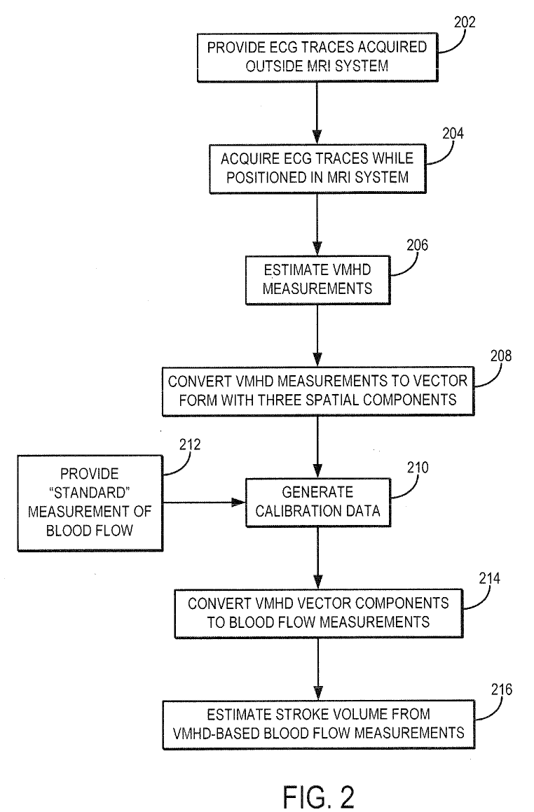

Referring now to FIG. 2, a flowchart is illustrated as setting forth the steps of an example method for estimating a quantitative measurement of left ventricular stroke volume in a subject from magnetohydrodynamic voltages generated by blood flowing in the subject's vasculature while the subject is positioned in an MRI scanner.

The method includes providing ECGs recorded from a subject when they were not positioned in MRI system, as indicated at step 202. These “real” ECG traces can be acquired immediately before placing the subject in the MRI system, or some other duration of time before placing the subject in the MRI system. ECGs are then recorded while the subject is positioned in the MRI system, as indicated at step 204. As one example, the ECGs can be recorded using an MRI-compatible 12-lead recording system, such as the one described in co-pending U.S. Patent Application No. US 2014/0171783, which is herein incorporated by reference in its entirety. In other examples, fewer leads can also be used. Measurements of VMHD at each electrode are then estimated from the provided ECGs, as indicated at step 206. These voltages can be extracted from each electrode by subtracting the ECGs recorded from the subject outside of the MRI system and the ECGs recorded while the subject is positioned in the MRI system.

The VMHD measurements are then converted to a vector form, as indicated at step 208. As one example, an inverse Dower transform can be used to convert the VMHD traces into a vectorcardiogram (“VCG”) frame of reference, where VMHD is composed of three spatial components along the x-direction, y-direction, and z-direction, which are defined by the axes of the MRI system such that the B₀ field is oriented along the z-direction,

VMHD(t)_{VCG}=[VMHD(t)_{X}VMHD(t)_{Y}VMHD(t)_{Z}] (2).

In this configuration, the x-direction corresponds to the left-right direction and the y-direction corresponds to the up-down direction. If a subject is positioned in the MRI scanner in a head first supine orientation, then the x-axis will correspond to the left-right (L-R) direction, the y-axis will correspond to the anterior-posterior (A-P) direction, and the z-axis will correspond to the superior-inferior (S-I) direction.

More generally, the VMHD vector can include components in a one-dimensional array, and these components can express VMHD measurements in terms of combinations of electrocardiogram and vectorcardiogram reference frames. The VMHD measurements can thus also be converted to a more generalized form that can be expanded or reduced to n total terms that include any suitable combination of VCG or ECG traces,

$$VMHD ( t ) = {\sum }_{i = 1}^{n} {VMHD ( t )}_{i} . \qquad ( 3 )$$

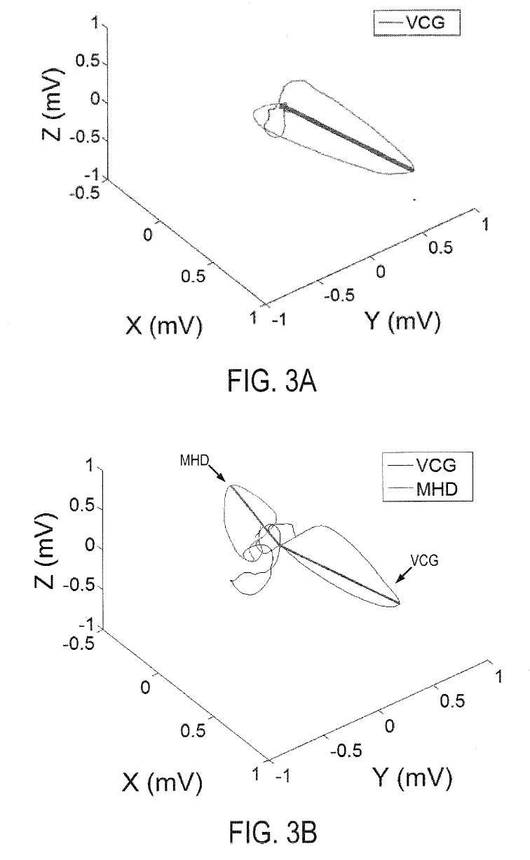

The inverse Dower transform uses the independent VMHD traces from the electrode leads to produce the vector components, VMHD_{X}, VMHD_{Y}, and VMHD_{Z}. An example vectorcardiogram obtained from a subject not positioned in an MRI system is illustrated in FIG. 3A, and an example vectorcardiogram obtained from the same subject while positioned in an MRI system is illustrated in FIG. 3B. Because a VCG provides vectorial information, it provides a frame of reference for describing the heart's electrical activity during the development of the multiple-parameter linear regression (“MLR”) model for correlating VMHD measurements to blood flow.

Referring again to FIG. 2, the spatial components of the VMHD vector are then calibrated against a standard measurement of blood flow to generate calibration data, as indicated at step 210. In some embodiments, spatial components of the ECG traces can also be calibrated against the standard measurement of blood flow. Thus, a standard measure of blood flow in the subject is provided, as indicated at step 212. As an example, the VMHD components are calibrated against a measurement of blood flow derived from cine (multiple cardiac phase) phase contrast MRI, which is a “gold standard” for estimating blood flow in the clinic. A linear relationship can be assumed between the standard flow measurement, Flow(t), and the VMHD vector components as follows,

Flow(t)=A₀+A₁·VMHD(t)_{X}+A₂·VMHD(t)_{Y}+A₃·VMHD(t)_{Z} (4).

Calibration can thus include fitting the VMHD vector components and the standard blood flow measurement to the MLR model of Eqn. (4) to obtain the subject-specific coefficients, A₀, A₁, A₂, and A₃. Eqn. (4) can also be generalized as follows,

$$Flow ( t ) = {A}_{0} + {\sum }_{i = 1}^{n} {A}_{i} {VMHD ( t )}_{i} ; \qquad ( 5 )$$

where i is the number of components used.

The calibration data are subsequently used to convert the VMHD vector components into blood flow measurements, as indicated at step 214. As an example, the subject-specific coefficients can be used to correlate the VMHD vector components to blood flow as a function of time using the equation of fit represented in Eqn. (4). For instance, VMHD-based measurements of blood flow can be estimated by inputting the VMHD vector components and subject-specific coefficients into Eqn. (4).

Quantitative measurements of SV are then computed from the VMHD-based measurements of blood flow, as indicated at step 216. Advantageously, after the rapid calibration described above is performed, the quantitative SV measurements can be continuous measurements that are provided in real-time, and can be provided for the entire duration that subject is in the bore of the MRI scanner. SV, which is the volume of flow ejected from the left ventricle over the systolic phase of the cardiac cycle, can be expressed as,

$$SV = {\int }_{Systole} {Flow}_{VMHD} ( t ) dt ; \qquad ( 6 )$$

where Flow_{VMHD}(t) is the VMHD-based blood flow measurement computed from the VMHD components using the calibration data (i.e., the subject-specific coefficients).

Thus, the systems and methods described here can provide a non-invasive, continuous, real-time quantitative measurements of both blood flow volume and stroke volume from VMHD measurements. Particularly, these parameters can be measured while the subject is positioned in an MRI system and while they are undergoing imaging or an image-guided intervention. Advantageously, the systems and methods described here provide physiological monitoring of left ventricular function, which is important for MRI of patients at risk and MRI-guided intervention on patients at risk.

As an example, the American Heart Association, American College of Cardiology, and the American Association of Anesthesiologists all recommend continuous monitoring of SV during surgery or other interventions in the following patient populations and situations: (1) mildly hypertensive patients, and especially those with preoperative organs injuries, such as those with ischemic damage where the risk of organ damage or morbidity during surgery is elevated; (2) situations with inaccurate non-invasive blood pressure readings (e.g., arrhythmia conditions including atrial fibrillation and premature ventricular contractions), where cine phase-contrast MRI will not be accurate; and (3) when rapid changes in blood pressure are expected during surgery (e.g., cardiovascular instability). As a result, implementation of the systems and methods described here allow such patients to be examined and treated, which can greatly enlarge the patient populations accepted for MRI or MRI-guided therapeutic procedures.

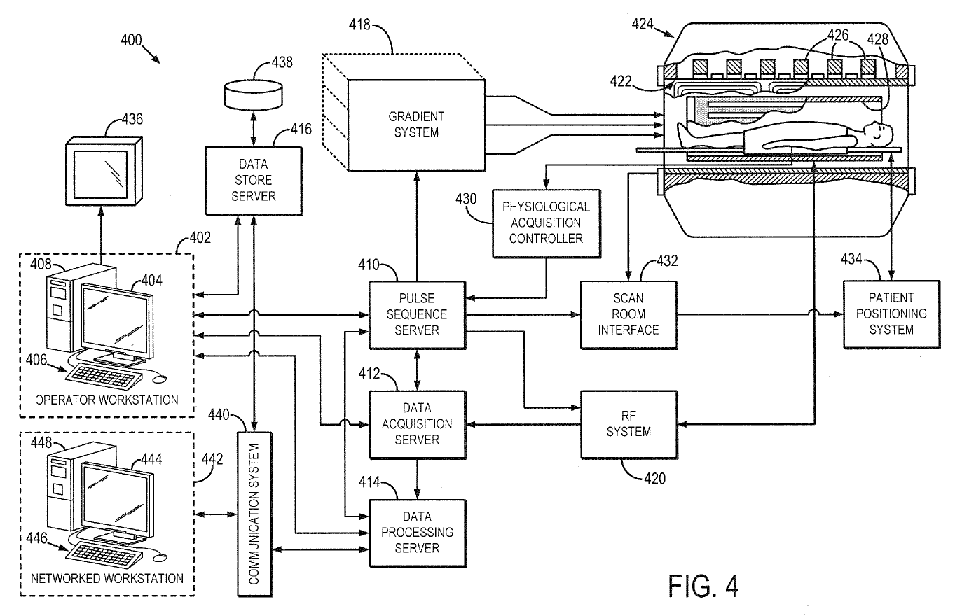

Referring particularly now to FIG. 4, an example of a magnetic resonance imaging (“MRI”) system 400 is illustrated. The MRI system 400 includes an operator workstation 402, which will typically include a display 404; one or more input devices 406, such as a keyboard and mouse; and a processor 408. The processor 408 may include a commercially available programmable machine running a commercially available operating system. The operator workstation 402 provides the operator interface that enables scan prescriptions to be entered into the MRI system 400. In general, the operator workstation 402 may be coupled to four servers: a pulse sequence server 410; a data acquisition server 412; a data processing server 414; and a data store server 416. The operator workstation 402 and each server 410, 412, 414, and 416 are connected to communicate with each other. For example, the servers 410, 412, 414, and 416 may be connected via a communication system 440, which may include any suitable network connection, whether wired, wireless, or a combination of both. As an example, the communication system 440 may include both proprietary or dedicated networks, as well as open networks, such as the internet.

The pulse sequence server 410 functions in response to instructions downloaded from the operator workstation 402 to operate a gradient system 418 and a radiofrequency (“RF”) system 420. Gradient waveforms necessary to perform the prescribed scan are produced and applied to the gradient system 418, which excites gradient coils in an assembly 422 to produce the magnetic field gradients Gₓ, G_{y}, and G_{z} used for position encoding magnetic resonance signals. The gradient coil assembly 422 forms part of a magnet assembly 424 that includes a polarizing magnet 426 and a whole-body RF coil 428.

RF waveforms are applied by the RF system 420 to the RF coil 428, or a separate local coil (not shown in FIG. 4), in order to perform the prescribed magnetic resonance pulse sequence. Responsive magnetic resonance signals detected by the RF coil 428, or a separate local coil (not shown in FIG. 4), are received by the RF system 420, where they are amplified, demodulated, filtered, and digitized under direction of commands produced by the pulse sequence server 410. The RF system 420 includes an RF transmitter for producing a wide variety of RF pulses used in MRI pulse sequences. The RF transmitter is responsive to the scan prescription and direction from the pulse sequence server 410 to produce RF pulses of the desired frequency, phase, and pulse amplitude waveform. The generated RF pulses may be applied to the whole-body RF coil 428 or to one or more local coils or coil arrays (not shown in FIG. 4).

The RF system 420 also includes one or more RF receiver channels. Each RF receiver channel includes an RF preamplifier that amplifies the magnetic resonance signal received by the coil 428 to which it is connected, and a detector that detects and digitizes the I and Q quadrature components of the received magnetic resonance signal. The magnitude of the received magnetic resonance signal may, therefore, be determined at any sampled point by the square root of the sum of the squares of the I and Q components:

M=\sqrt{I²+Q²} (7);

and the phase of the received magnetic resonance signal may also be determined according to the following relationship:

$$ϕ = {tan}^{- 1} ( \frac{Q}{I} ) . \qquad ( 8 )$$

The pulse sequence server 410 also optionally receives patient data from a physiological acquisition controller 430. By way of example, the physiological acquisition controller 430 may receive signals from a number of different sensors connected to the patient, such as electrocardiograph (“ECG”) signals from electrodes, or respiratory signals from a respiratory bellows or other respiratory monitoring device. Such signals are typically used by the pulse sequence server 410 to synchronize, or “gate,” the performance of the scan with the subject's heart beat or respiration.

The pulse sequence server 410 also connects to a scan room interface circuit 432 that receives signals from various sensors associated with the condition of the patient and the magnet system. It is also through the scan room interface circuit 432 that a patient positioning system 434 receives commands to move the patient to desired positions during the scan.

The digitized magnetic resonance signal samples produced by the RF system 420 are received by the data acquisition server 412. The data acquisition server 412 operates in response to instructions downloaded from the operator workstation 402 to receive the real-time magnetic resonance data and provide buffer storage, such that no data is lost by data overrun. In some scans, the data acquisition server 412 does little more than pass the acquired magnetic resonance data to the data processor server 414. However, in scans that require information derived from acquired magnetic resonance data to control the further performance of the scan, the data acquisition server 412 is programmed to produce such information and convey it to the pulse sequence server 410. For example, during prescans, magnetic resonance data is acquired and used to calibrate the pulse sequence performed by the pulse sequence server 410. As another example, navigator signals may be acquired and used to adjust the operating parameters of the RF system 420 or the gradient system 418, or to control the view order in which k-space is sampled. In still another example, the data acquisition server 412 may also be employed to process magnetic resonance signals used to detect the arrival of a contrast agent in a magnetic resonance angiography (“MRA”) scan. By way of example, the data acquisition server 412 acquires magnetic resonance data and processes it in real-time to produce information that is used to control the scan.

The data processing server 414 receives magnetic resonance data from the data acquisition server 412 and processes it in accordance with instructions downloaded from the operator workstation 402. Such processing may, for example, include one or more of the following: reconstructing two-dimensional or three-dimensional images by performing a Fourier transformation of raw k-space data; performing other image reconstruction algorithms, such as iterative or backprojection reconstruction algorithms; applying filters to raw k-space data or to reconstructed images; generating functional magnetic resonance images; calculating motion or flow images; and so on.

Images reconstructed by the data processing server 414 are conveyed back to the operator workstation 402 where they are stored. Real-time images are stored in a data base memory cache (not shown in FIG. 4), from which they may be output to operator display 402 or a display 436 that is located near the magnet assembly 424 for use by attending physicians. Batch mode images or selected real time images are stored in a host database on disc storage 438. When such images have been reconstructed and transferred to storage, the data processing server 414 notifies the data store server 416 on the operator workstation 402. The operator workstation 402 may be used by an operator to archive the images, produce films, or send the images via a network to other facilities.

The MRI system 400 may also include one or more networked workstations 442. By way of example, a networked workstation 442 may include a display 444; one or more input devices 446, such as a keyboard and mouse; and a processor 448. The networked workstation 442 may be located within the same facility as the operator workstation 402, or in a different facility, such as a different healthcare institution or clinic.

The networked workstation 442, whether within the same facility or in a different facility as the operator workstation 402, may gain remote access to the data processing server 414 or data store server 416 via the communication system 440. Accordingly, multiple networked workstations 442 may have access to the data processing server 414 and the data store server 416. In this manner, magnetic resonance data, reconstructed images, or other data may be exchanged between the data processing server 414 or the data store server 416 and the networked workstations 442, such that the data or images may be remotely processed by a networked workstation 442. This data may be exchanged in any suitable format, such as in accordance with the transmission control protocol (“TCP”), the Internet protocol (“IP”), or other known or suitable protocols.

The operator workstation 402 or networked workstation 442 can be programmed to implement the methods for calculating continuous and real-time quantitative measurements of blood flow and stroke volume from VMHD measurements, as described above. In these instances, the operator workstation 402 or networked workstation 442 can be used to provide continuous real-time physiological monitoring of a subject during an MRI scan or an MRI-guided interventional procedure.

### EXAMPLE

### Continuous Rapid Quantification of Stroke Volume Using VMHD Measurements

An example study was conducted to investigate estimating blood-flow, as a function of time in the cardiac cycle, and left ventricular stroke volume from VMHD measurements extracted from intra-MRI ECGs using the methods described above. The method should allow for non-invasive beat-to-beat stroke volume estimation during clinical MR scanning and cardiac MRI stress testing. This non-invasive real-time physiological measure of patient condition can be used during conventional cardiac MRI routines, and could potentially replace invasive blood pressure (“IBP”) monitoring during complex interventional procedures.

### Methods

As described above, a patient-specific multiple-parameter linear regression (“MLR”) model was used to obtain a patient-specific approximation of volumetric blood-flow (in mL/s) using the correlation with extracted VMHD in a vectorcardiogram (“VCG”) reference frame (VMHD_{VCG}). A blood-flow “gold-standard”, as a function of time, was obtained using a cine phase contrast (“PC”) MRI scan in the aortic arch. VMHD_{VCG}-derived volumetric-blood-flow was then time-integrated over the systolic phase to estimate SV (SV_{MHD}), as described above. The success of the MHD-derived stroke volume, SV_{MHD}, and blood-flow, BF_{MHD}, was thereafter evaluated by comparison with results obtained from cine PC MRI. Cross-correlation between VMHD and MRI derived blood-flow was calculated to determine the fit's significance.

A GE Cardiolab-IT digital ECG recording system, modified to be MRI-compatible, was used to record 12-lead ECG traces using standard 12-lead ECG chest placement in three healthy volunteer subjects. A similar 12-lead ECG recording system, installed at another clinical site, was used to record 12-lead ECG traces in four healthy volunteer subjects. Conventional cine PC and real-time (single-cardiac-beat) PC (“RTPC”) was used to validate VMHD_{VCG}-based metrics for each test subject.

### RESULTS

MRI Training and Validation. An equation of fit was developed using patient-specific coefficients for each subject in this test series, derived using conventional cine PC MRI taken during baseline heart rate (Table 1).

TABLE 1
Multiple Linear Regression for Blood-flow
and Stroke Volume Estimation using VMHD_{VCG}

| Subjects | Cross-Correlation | SV_{VMHD} | SV_{PC} | Initial Fit Error |
|---|---|---|---|---|
| 1 | 0.94 | 73.7 mL | 75.7 mL | 2.62% |
| 2 | 0.95 | 78.5 mL | 78.1 mL | 0.59% |
| 3 | 0.84 | 55.1 mL | 53.2 mL | 3.56% |
| 4 | 0.88 | 84.8 mL | 84.8 mL | 0.09% |
| 5 | 0.89 | 71.1 mL | 71.7 mL | 0.86% |
| 6 | 0.95 | 79.0 mL | 78.4 mL | 0.76% |
| (50^{th}) | | | | |
| 7 | 0.99 | 76.8 mL | 77.6 mL | 1.00% |
| (90^{th}) | | | | |

The equation of fit was used to transform VMHD over time to aortic blood-flow velocity. Subject VMHD-derived blood flow and SV were compared to phase contrast cine MRI results to evaluate fit, with correlation determined through a Spearman's Ranked Coefficient, found to be greater than 0.84. VMHD-based SV was determined with a less than 5 percent error as compared to PC MRI in all subjects.

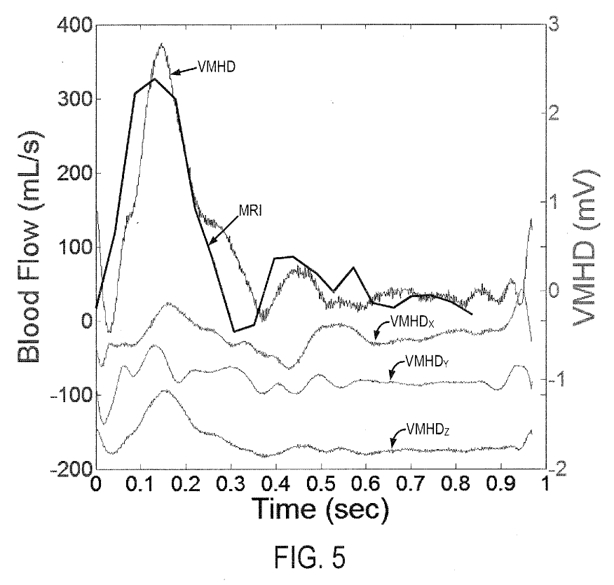

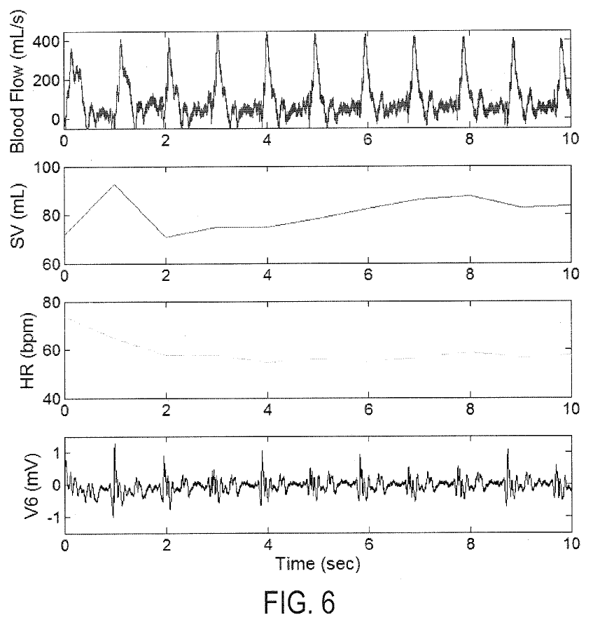

Conventional Cine PC MRI scans were obtained for each subject and used to extract blood-flow as a function of time. Through statistical analysis and experimental quantification of previously-described coefficients (A₀, A₁, A₂, and A₃) (Eqn. (4)), the patient-specific relationship between extracted VMHD_{VCG} and blood-flow BF_{MHD} was achieved, and an appropriate fit was computed. An example of MRI-based and VMHD-based measurements of blood flow are illustrated in FIG. 5. Using this methodology, real-time beat-to-beat BF_{MHD} and SV_{MHD}, as well as the associated heart rate, were estimated, as illustrated in FIG. 6, thereby providing continuous flow and SV monitoring.

Exercise Stress Testing. To validate the efficacy of VMHD-derived blood flow estimation, exercise stress tests were performed to compare changes in blood flow and SV during peak stress and after full relaxation heart rate levels, as recorded by RTPC MRI scans and VMHD_{VCG}-derived metrics. The robustness of the methods described above to variations in subject anthropometry, and electrode placement, were observed by comparing two from this study. One subject (subjects #6) was chosen as an accurate representation of a 50th Percentile Male (Weight: 68 kg; Height: 168 cm; Chest circumference: 94 cm) in the population as concerns both height and weight, and the other (subject #7) was chosen as an accurate representation of a 90th Percentile Male (Weight: 127 kg; Height: 185 cm; Chest circumference: 135 cm) in the population as concerns both height and weight. VMHD_{VCG}-derived blood-flow related metrics were quantified for each subject during an exercise stress test sequence to evaluate the performance across all subjects (Table 2).

TABLE 2
Assessment of VMHD_{VCG}-derived Flow Metrics in Relationship to MRI

| | Stress Testing | | | | Return to Baseline | | | |
|---|---|---|---|---|---|---|---|---|
| | Flow | Peak | Ejection | | BF- | Peak | Ejection | |
| | Waveform | Flow | Period | | Waveform | Flow | Period | SV |
| # | Correlation | Error | Difference | SV Error | Correlation | Error | Difference | Error |
| 4 | 0.85 | 6.03% | 26 ms | 10.35% | 0.78 | 1.17% | 80 ms | 0.07% |
| 5 | 0.94 | 2.90% | 45 ms | 11.15% | 0.85 | 5.98% | 54 ms | 8.86% |
| 6 | 0.94 | 11.31% | 20 ms | 6.30% | 0.93 | 8.07% | 50 ms | 2.08% |
| 7 | 0.97 | 0.66% | 40 ms | 2.30% | 0.99 | 1.98% | 50 ms | 5.54% |
| Mean | 0.93 | 5.23% | 38 ms | 7.53% | 0.89 | 4.30% | 59 ms | 4.14% |
| Max | 0.97 | 11.31% | 56 ms | 11.15% | 0.99 | 8.07% | 80 ms | 8.86% |

### DISCUSSION

Non-invasive beat-to-beat stroke volume and blood flow velocity estimates were obtained from MHD voltages extracted from 12-lead ECGs, and then cast into the VCG frame-of-reference. This provided a technique for enhanced patient monitoring inside the bore of an MRI scanner, requiring only a relatively short cine PC MRI calibration (about 20 seconds in duration) to provide the required patient-specific parameters prior to continuous monitoring during the remaining duration a subject was positioned in the bore of the MRI system.

An average error of 7.53 percent and 4.14 percent in VMHD_{VCG}-derived SV estimation, respectively, as compared to the RTPC CINE estimate was found during peak stress and after the full relaxation. The mean predicted ejection period was shown to differ from the RTPC CINE by an average of 38 ms during peak stress, and by an average of 59 ms after the full relaxation. Average BF waveform correlation between the PC and MHD methods decreased from 0.93 to 0.89 after full relaxation. VMHD_{VCG}-derived estimates were shown to maintain an average error of less than 8 percent in all cases, with a marginal decrease in accuracy after full relaxation.

Beat-to-beat SV_{MHD} has increased temporal resolution relative to cine PC and RTPC SVPC, even when employing RTPC (0.5 ms resolution versus 40 ms resolution). Furthermore, use of MRI for continuously monitoring SV is impractical due because the scanner is needed to image additional contrasts or other regions of the heart.

It is contemplated that PC pre-scans can be used to train an active Kalman filter for VMHD-derived estimates to further increase the accuracy of blood flow and SV estimates. The addition of secondary MRI-compatible physiological monitoring devices, such as a pulse oximeter, would also provide an enhanced level of information and potentially lead to an increase in method accuracy when such information is included into the fitting equation.

VMHD-derived SV and blood flow estimates allow for accurate, non-invasive, real-time cardiovascular monitoring during MRI-guided surgical procedures and interventions. This method could be integrated into the clinical workflow, and installed into existing ECG recording systems, requiring a simple software upgrade.

The present invention has been described in terms of one or more preferred embodiments, and it should be appreciated that many equivalents, alternatives, variations, and modifications, aside from those expressly stated, are possible and within the scope of the invention.

1. A method for computing a measurement of loft ventricular stroke volume from a subject positioned in a bore of a magnetic resonance imaging (MRI) system, the steps of the method comprising:
(a) recording electrocardiogram (ECG) measurements from a subject positioned in a bore of an MRI system;
(b) estimating magnetohydrodynamic voltage (VMHD) measurements from the ECG measurements;
(c) generating a VMHD vector by converting the VMHD measurements to a vectorcardiogram reference frame;
(d) generating calibration data by correlating the VMHD vector to a standard measure of blood flow obtained from the subject;
(e) generating a VMHD-based blood flow measurement by converting the VMHD vector to the VMHD-based blood flow measurement using the calibration data; and
(f) computing a stroke volume measurement from the VMHD-based blood flow measurement.

2. The method as recited in claim 1, wherein step (b) includes providing to the computer system, ECG measurements acquired from the subject when the subject was not positioned in the bore of the MRI system and estimating the VMHD measurements by computing a difference between the ECG measurements recorded in step (a) and the provided ECG measurements acquired from the subject when the subject was not positioned in the bore of the MRI system.

3. The method as recited in claim 1, wherein step (c) includes generating the VMHD vector by performing an inverse Dower transform on the VMHD measurements, wherein the VMHD vector includes three spatial components and each spatial component is associated with one of an x-direction, a y-direction, and a z-direction such that the z-direction corresponds to a longitudinal axis of the MRI system.

4. The method as recited in claim 1, wherein the standard measure of blood flow is a measurement of blood flow estimated from cine phase contrast magnetic resonance images acquired from the subject.

5. The method as recited in claim 1, wherein step (d) includes fitting the VMHD vector and the standard measure of blood flow to a multiple-parameter linear regression (MLR) model to generate the calibration data as subject-specific coefficients for the MLR model.

6. The method as recited in claim 5, wherein step (e) includes generating the VMHD-based blood flow measure by inputting the subject-specific coefficients and the VMHD vector into the MLR model.

7. The method as recited in claim 1, wherein step (f) includes computing the stroke volume measurement by integrating VMHD-based blood flow measurements generated during a systolic phase of the subject's cardiac cycle.

8. The method as recited in claim 1, wherein the VMHD vector generated in step (c) express the VMHD measurements in terms of combinations of electrocardiogram and vectorcardiogram reference frames.

9. A method for providing continuous real-time monitoring of left ventricular stroke volume in a subject positioned in a bore of a magnetic resonance imaging (MRI) system, the steps of the method comprising:
(a) providing a calibrated multiple-parameter linear regression (MLR) model to a computer system, wherein the MLR model includes subject-specific coefficients that relate magnetohydrodynamic voltage (VMHD) vectorcardiogram components to blood flow as a function of time;
(b) recording electrocardiogram (ECG) measurements from a subject positioned in a bore of an MRI system;
(c) estimating VMHD measurements from the ECG measurements;
(d) generating VMHD vectorcardiogram components by converting the VMHD measurements to a vectorcardiogram reference frame;
(e) generating VMHD-based blood flow measurements by inputting the VMHD vectorcardiogram components to the calibrated MLR model; and
(f) computing stroke volume measurements from the VMHD-based blood flow measurements, thereby providing continuous real-time monitoring of left ventricular stroke volume in the subject while the subject is positioned in the bore of the MRI system.

10. The method as recited in claim 9, wherein step (c) includes providing to the computer system, ECG measurements acquired from the subject when the subject was not positioned in the bore of the MRI system and estimating the VMHD measurements by computing a difference between the ECG measurements recorded in step (b) and the provided ECG measurements acquired from the subject when the subject was not positioned in the bore of the MRI system.

11. The method as recited in claim 9, wherein step (d) includes generating the VMHD vectorcardiogram components by performing an inverse Dower transform on the VMHD measurements.

12. The method as recited in claim 11, wherein the VMHD vectorcardiogram components comprise a first spatial component associated with an x-direction defined relative to the bore of the MRI system, a second spatial component associated with a y-direction defined relative to the bore of the MRI system, and a third spatial component associated with a z-direction defined as a longitudinal axis of the bore of the MRI system.

13. The method as recited in claim 9, wherein step (f) includes computing the stroke volume measurements by integrating VMHD-based blood flow measurements generated during a systolic phase of the subject's cardiac cycle.

14. The method as recited in claim 9, wherein step (a) includes providing a standard measure of blood flow obtained from the subject and forming the MLR model by fitting the standard measure of blood flow and a set of VMHD vectorcardiogram components to a linear function that relates blood flow to the VMHD vectorcardiogram components through the subject-specific coefficients.

15. The method as recited in claim 14, wherein the standard measure of blood flow is computed from cine phase contrast magnetic resonance images acquired from the subject.
